(19) United States
(12) Patent Application Publication
(10) Pub. No.: US 2019/0094170 A1
(43) Pub. Date: Mar. 28, 2019

(54) ANALYTICAL TEST STRIP WITH INTEGRATED ELECTRICAL RESISTOR

(71) Applicant: Cilag GmbH International, Zug (CH)

(72) Inventor: David ELDER, Inverness (GB)

(21) Appl. No.: 15/712,636

(22) Filed: Sep. 22, 2017

(51) Int. Cl. G01N 27/327; A61B 5/05; C12Q 1/00

(52) U.S. Cl. CPC G01N 27/3272; C12Q 1/006; G01N 27/3273; A61B 5/05

(57) ABSTRACT

An electrochemical-based analytical test strip for the determination of an analyte (such as glucose) in a bodily fluid sample (e.g., a whole blood sample) includes an electrically-insulating substrate layer, and a first electrically-conductive layer disposed on the electrically-insulating substrate layer that includes a first electrode portion and a first electrical contact pad. The electrochemical-based analytical test strip also includes a patterned spacer layer disposed on the first electrically-conductive layer, an electrically-insulating top layer with an underside surface disposed above the patterned spacer layer and a second electrically-conductive layer disposed on the underside surface of the electrically-insulating top layer. Moreover, the second electrically-conductive layer includes a second electrode portion and a second electrical contact pad. The electrochemical-based analytical test strip further includes an integrated resistor configured as an electrically conductive path of predetermined resistance between the first electrically conductive layer and the second electrically conductive layer.

### BACKGROUND OF THE INVENTION

### Field of the Invention

The present invention relates, in general, to medical devices and, in particular, to analytical test strips and related methods.

### Description of Related Art

The determination (e.g., detection and/or concentration measurement) of an analyte in, or a characteristic of, a fluid sample is of particular interest in the medical field. For example, it can be desirable to determine glucose, ketone bodies, cholesterol, lipoproteins, triglycerides, acetaminophen, hematocrit and/or HbA1c concentrations in a sample of a bodily fluid such as urine, blood, plasma or interstitial fluid. Such determinations can be achieved using analytical test strips, based on, for example, visual, photometric or electrochemical techniques. Conventional electrochemical-based analytical test strips are described in, for example, U.S. Pat. Nos. 5,708,247, and 6,284,125, each of which is hereby incorporated in full by reference.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention, in which:

FIG. 1 is a simplified perspective view of an electro-chemical based analytical test strip according to an embodiment of the present invention;

FIG. 2 is a simplified exploded view of the electrochemical-based analytical test strip of FIG. 1;

FIG. 3 is a further simplified exploded view of a portion of the electrochemical-based analytical test strip of FIG, 1;

FIGS. 4A and 4B are a simplified bottom view of the electrochemical-based analytical test strip of FIG. 1 simplified and a simplified electrical schematic diagram depicting an integrated resistance and a cell impedance of the electrochemical-based analytical test strip;

FIG. 5 is a simplified top view of the electrochemical-based analytical test strip of FIG. 1 operably inserted in an associated meter (M);

FIG. 6 is a simplified exploded view of another electrochemical-based analytical test strip according to an embodiment of the present invention; and

FIG. 7 is a flow diagram depicting stages in a method for determining an analyte in a bodily fluid sample according to an embodiment of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein.

Electrochemical-based analytical test strip for the determination of an analyte (such as glucose) in, or a characteristic of, a bodily fluid sample (for example, a whole blood sample) according to embodiments of the present invention include an electrically-insulating substrate layer, a first electrically conductive layer disposed on the electrically insulating substrate layer, a patterned spacer layer disposed on the first electrically conductive layer, an electrically insulating top layer with an underside surface disposed above the patterned spacer layer, and a second electrically conductive layer disposed on that underside surface. The electrochemical-based analytical test strip also includes an integrated resistor configured as an electrically conductive path of predetermined resistance between the first electrically conductive layer and the second electrically conductive layer.

Electrochemical-based analytical test strips according to embodiments of the present invention are beneficial in that, for example, the predetermined resistance of the integrated resistor can be employed to identify the electrochemical-based analytical test strip, an appropriate determination algorithm(s) to employ therewith, and appropriate electrical biasing during use of the electrochemical-based analytical test strip with a hand-held test meter. They are also beneficial in that they enable existing hand-held test meters to be used with a variety of electrochemical-based analytical test strips (i.e., a variety of electrochemical-based analytical test strips according to the present invention, each of a different predetermined resistance) as long as the hand-held test meter has been updated with the appropriate algorithm(s). Since algorithms stored in hand-held test meters can be updated remotely, hand-held test meters in the field can be updated and employed with newly introduced electrochemical-based analytical test strips according to the present invention.

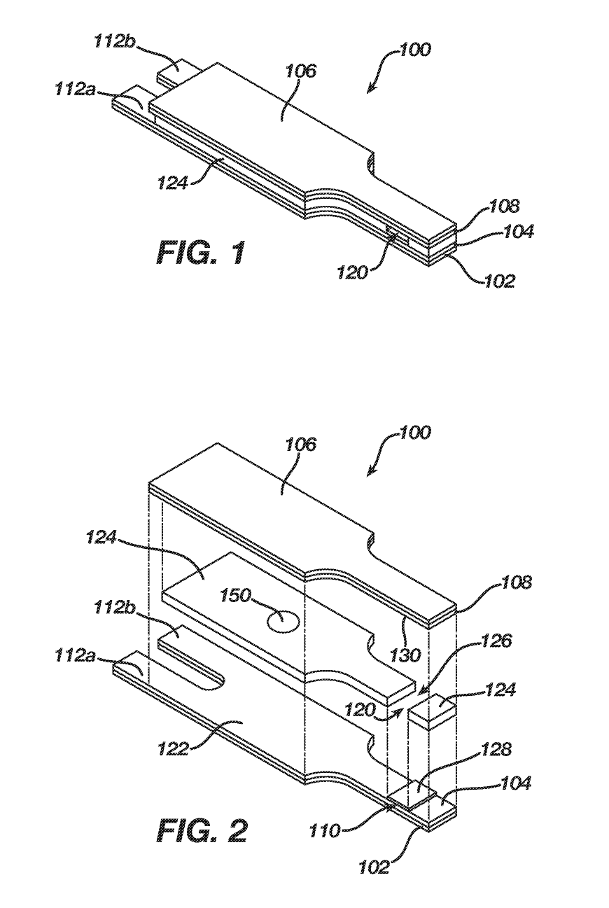

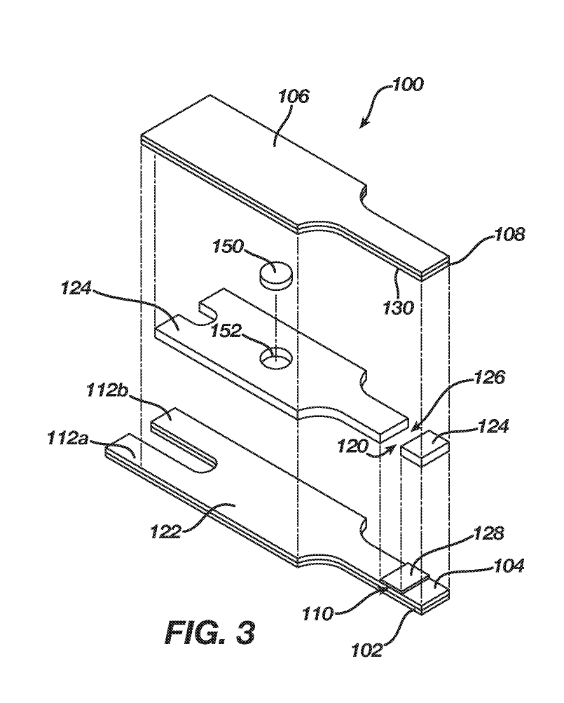

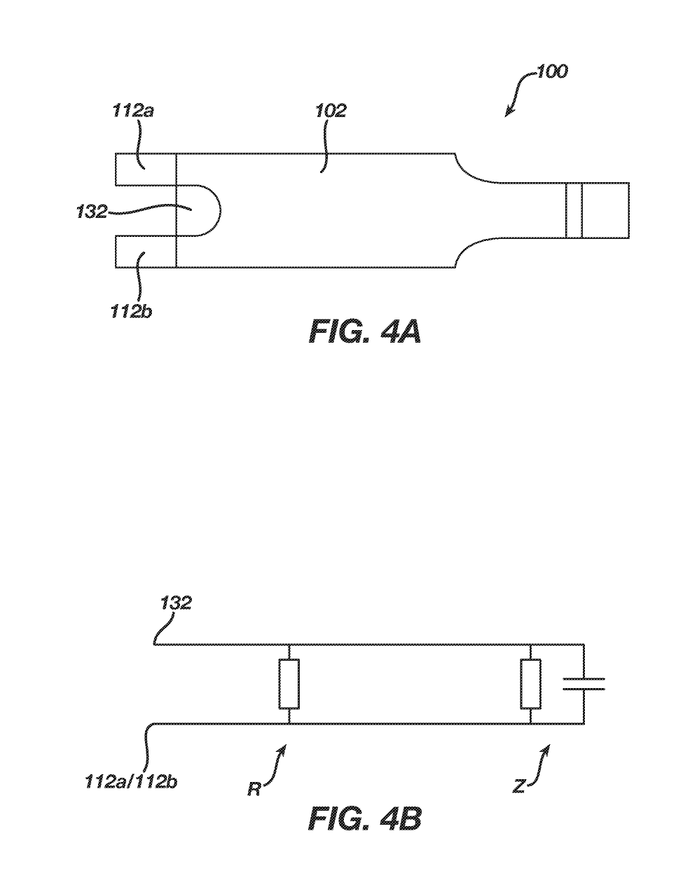

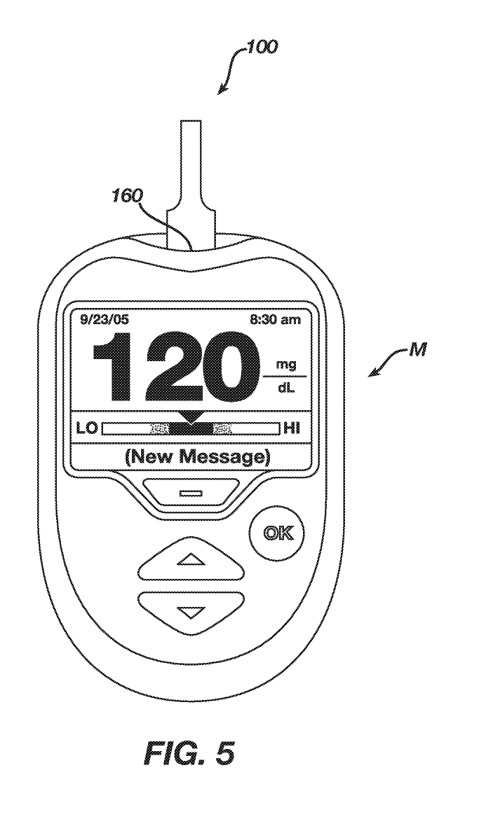

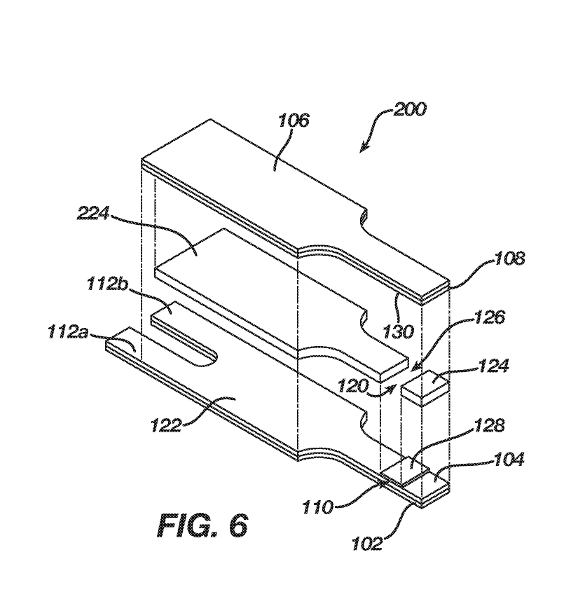

FIG. 1 is a simplified perspective view of an electrochemical-based analytical test strip 100 according to an embodiment of the present invention. FIG. 2 is a simplified exploded view of electrochemical-based analytical test strip 100. FIG. 3 is a further simplified exploded view of a portion of electrochemical-based analytical test strip 100. FIGS. 4A and 4B are a simplified bottom view of electrochemical-based analytical test strip 100 (FIG. 4A) and a simplified electrical schematic diagram depicting an integrated electrical resistance (R) and a cell impedance (Z composed of a resistor element and capacitor element in parallel) of the electrochemical-based analytical test strip (FIG. 4B). FIG. 5 is a simplified top view of electrochemical-based analytical test strip 100 operably inserted in an associated hand-held test meter (M). FIG. 6 is a simplified exploded view of another electrochemical-based analytical test strip 200 according to an embodiment of the present invention

Referring to FIGS. 1-6, electrochemical-based analytical test strip 100 for the determination of an analyte (such as glucose) in a bodily fluid sample (for example, a whole blood sample) includes an electrically insulating substrate layer 102, and a first electrically conductive layer 104 disposed on electrically insulating substrate layer 102. First electrically conductive layer 104 includes a first electrode portion 110 and first electrical contact pads 112a and 112b.

Electrochemical-based analytical test strip 100 also includes an enzymatic reagent layer 128 and a patterned spacer layer 124 disposed on first electrically conductive layer 104. In addition, electrochemical-based analytical test strip 100 includes an electrically insulating top layer 106 with an underside surface (not visible in the perspective of the FIGs.) disposed above patterned spacer layer 124 and a second electrically conductive layer 108 disposed on underside surface of electrically insulating top layer 106.

Second electrically conductive layer 108 includes a second electrode portion 130 (see FIG. 2) and a second electrical contact pad 132 (see, in particular, FIG. 4A). In addition, patterned spacer layer 124 defines a sample-receiving chamber 126, having a sample application opening 120.

Electrochemical-based analytical test strip 100 also includes an integrated resistor 150 configured as an electrically conductive path of predetermined resistance between the first electrically conductive layer 104 and the second electrically conductive layer 108. Integrated resistor 150 can be formed of any suitable electrically resistive material, for example, an electrically resistive material that with an electrical resistance in the range of 1.0 k-ohm to 10 m-ohm. Suitable electrically resistive materials include silicon rubber loaded with carbon (available commercially from RD Rubber Technology, Santa Fe Springs, Calif., USA and Advanced Rubber Products, Wyoming, N.Y., USA).

As depicted in FIG. 3, integrated resistor 150 can be, for example, cylindrical in shape with a circular cross-section and disposed in an aperture 152 that has punched into patterned spacer layer 124 during the manufacturing of electrochemical-based analytical test strip 100. If desired, integrated resistor 150 can be electrically contacted to first electrically conductive layer 104 and second electrically conductive layer 108 using a suitable electrically conductive adhesive. A typical, but non-limiting, diameter of integrated resistor 150 can be, for example, in the range of 1.5 mm to 2.5 mm.

The simplified electrical schematic of FIG. 4B depicts how the presence of integrated resistor 150 creates a predetermined electrical resistance (R) in parallel with the cell impedance of the sample chamber. The inherent resistance and capacitance of the sample receiving chamber and any bodily fluid sample therein (which is the electrically represented as an impedance) are also depicted in FIG. 4B. In this regard, the predetermined electrical resistance of integrated resistor 150 can be thought of as an artificial resistance in that it has been inserted (i.e. integrated) into the electrochemical-based analytical test strip for test strip identification purposes and is not an electrical resistance otherwise inherent in a sample chamber filled with a bodily fluid sample.

FIG. 6 depicts an alternative embodiment of an electrochemical-based analytical test strip according to the present invention. FIG. 6 depicts an electrochemical-based analytical test strip 200 wherein like elements with electrochemical-based analytical test strip 100 are labeled with like numerals. The difference between the embodiments of FIG. 6 and FIGS. 1-5 is that the patterned spacer layer 224 of electrochemical-based analytical test strip 200 is slightly conductive and thus configured as an integrated resistor. Such an electrically resistive patterned spacer layer can be formed of any suitable material including, for example, an electrically conductive resin (available commercially as LNP Stat-kon Electrically Conductive Resin—VCF2020″ from SABIC, Pittsfield, Mass., USA). A typical thickness for patterned spacer layer 124 is, for example, 95 microns.

Returning to FIGS. 1-5, electrically-insulating substrate layer 102 and electrically insulating top layer 106 can be formed of any suitable material known to one skilled in the art including, for example, a nylon substrate, polycarbonate substrate, a polyimide substrate, a polyvinyl chloride substrate, a polyethylene substrate, a polypropylene substrate, a glycolated polyester (PETG) substrate, or a polyester substrate.

First and second electrically conductive layers 104 and 108 can be formed of any suitable conductive material including, for example, sputter deposited palladium and sputter deposited gold. Electrically conductive layers 104 and 108 can have any suitable thickness. For example, the thickness of electrically conductive layers formed via sputtering or gold (Au) or palladium (Pd) can be approximately 15 nm.

Patterned spacer layer 124 can be formed, for example, from a thin plastic, typically 50 microns in thickness, with a 22.5-micron electrically conductive adhesive layer coated on each side resulting in a total thickness of approximately 95 microns.

Moreover, first electrode portion 110 and second electrode portion 130 are disposed in sample-receiving chamber 126 such that electrochemical-based analytical test strip 100 is configured for the determination of an analyte (such as glucose) in a bodily fluid sample (for example, a whole blood sample) that has filled the sample-receiving chamber 126.

Enzymatic reagent layer 128 can include any suitable enzymatic reagents, with the selection of enzymatic reagents being dependent on the analyte to be determined. For example, if glucose is to be determined in a blood sample, enzymatic reagent layer 128 can include a glucose oxidase or glucose dehydrogenase along with other components necessary for functional operation. Enzymatic reagent layer 128 can include, for example, glucose oxidase, tri-sodium citrate, citric acid, polyvinyl alcohol, hydroxyl ethyl cellulose, potassium ferrocyanide, antifoam, cabosil, and water. Further details regarding enzymatic reagent layers, and electrochemical-based analytical test strips in general, are in U.S. Pat. Nos. 6,241,862 and 6,733,655, the contents of which are hereby fully incorporated by reference.

Electrochemical-based analytical test strip 100 can be manufactured, for example, by the sequential aligned formation and integration of each of the aforementioned layers and integrated resistor. Any suitable techniques known to one skilled in the art can be used to accomplish such sequential aligned formation, including, for example, web-based techniques, screen printing, photolithography, photogravure, chemical vapour deposition and tape lamination techniques.

Once apprised of the present disclosure, one of skill in the art will recognize that the embodiment of FIGS. 1-5 depicts an electrochemical-based analytical test strip with a co-facial electrode configuration and that embodiments of electrochemical-based analytical test strips according to the present invention with a co-planar electrode configuration are also feasible. For example, an electrochemical-based analytical test strip for the determination of an analyte in a bodily fluid sample according to the present invention can include first and second electrically-conductive layers separated by a spacer layer and an integrated resistor configured as an electrically conductive path of predetermined resistance disposed between the first electrically conductive layer and the second electrically conductive layer.

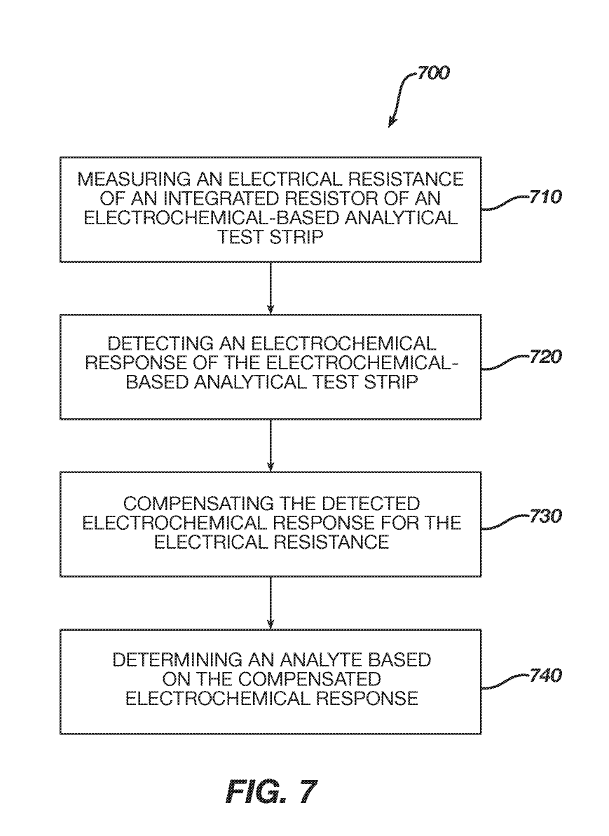

FIG. 7 is a flow diagram depicting stages in a method 700 for determining an analyte (such as glucose) in a bodily fluid sample (for example, a whole blood sample) according to an embodiment of the present invention. Method 700 includes (see step 710 of FIG. 7) measuring an electrical resistance of an integrated resistor of an electrochemical-based analytical test strip using a direct current (DC) bias. In step 710, the integrated resistor is configured as an electrically conductive path of predetermined electrical resistance between a first electrically conductive layer and a second electrically conductive layer of the electrochemical-based analytical test strip.

At step 720 of method 700, an electrochemical response (for example, a transient electrochemical response) of the electrochemical-based analytical test strip is detected following application of a bodily fluid sample to the electrochemical-based analytical test strip. In method 700, the application of the bodily fluid sample was after step 710 and prior to step 720.

In addition, at step 730, the detected electrochemical response is compensated for the electrical resistance of the integrated resistor thereby creating a compensated electrochemical response. Subsequently, at step 740 determining an analyte in the bodily fluid sample based on the compensated electrochemical response.

During method 700, the maximum electrical current through the integrated resistor can be any suitable maximum current such as, for example, 6 micro-amps. The measuring step of methods according to embodiments of the present invention can include applying a DC bias, measuring a resulting current and calculating the electrical resistance based on the applied DC bias and the resulting current.

The compensating step of methods according to embodiments of the present invention can include applying a compensation that is based on an applied voltage bias employed during the detecting step. For example, when the detecting step employs a plurality of applied voltage biases, the compensating step applies a plurality of compensations wherein each of the plurality of compensations corresponds to a different applied voltage bias. In such a circumstance, a transient electrochemical response can be compensated by subtracting the current flowing the integrated resistor at each different voltage bias level. For example, for a 100 k-ohm integrated resistor, at +20 mV bias an artificial current of 200 nA is subtracted, and at +300 mV of bias, a 3 micro-A bias is subtracted.

Moreover, method 700 can, if desired, include the step of selecting an algorithm for at least one of the detecting, compensating and determining steps based on the measured electrical resistance. Such a selected algorithm can include an algorithm that displays an error message on the hand-held test meter (such as hand-held test meter M of FIG. 5) should the measured electrical resistance indicate that the electrochemical-based analytical test strip is not compatible with the hand-held test meter.

Once apprised of the present disclosure, one skilled in the art will recognize that method 700 can be readily modified to incorporate any of the techniques, benefits, features and characteristics of electrochemical-based analytical test strips according to embodiments of the present invention and described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and methods within the scope of these claims and their equivalents be covered thereby.

1. An electrochemical-based analytical test strip for the determination of an analyte in a bodily fluid sample, the electrochemical-based analytical test strip comprising:
an electrically-insulating substrate layer;
a first electrically-conductive layer disposed on the electrically-insulating substrate layer, the first electrically-conductive layer including:
a first electrode portion; and
a first electrical contact pad;
a patterned spacer layer disposed on the first electrically-conductive layer;
an electrically-insulating top layer with an underside surface disposed above the patterned spacer layer;
a second electrically-conductive layer disposed on the underside surface of the electrically-insulating top layer, the second electrically-conductive layer including:
a second electrode portion; and
a second electrical contact pad; and
an integrated resistor configured as an electrically conductive path of predetermined resistance between the first electrically conductive layer and the second electrically conductive layer,
wherein the patterned spacer layer defines a sample-receiving chamber containing the first electrode portion and the second electrode portion.

2. The electrochemical-based analytical test strip of claim 1 wherein the integrated resistor has an electrical resistance in the range of 1.0 k-ohm to 10 M-ohm.

3. The electrochemical-based analytical test strip of claim 1 wherein the integrated resistor is disposed through the pattered spacer layer,

4. The electrochemical-based analytical test strip of claim 3 wherein the integrated resistor is cylindrical in shape.

5. The electrochemical-based analytical test strip of claim 4 wherein a diameter of the integrated resistor is in the range of 1.5 mm to 2.5 mm.

6. The electrochemical-based analytical test strip of claim 3 wherein the patterned spacer layer is essentially electrically-non-conductive.

7. The electrochemical-based analytical test strip of claim 3 wherein the integrated resistor is formed of a combination of at least silicon rubber and carbon.

8. The electrochemical-based analytical test strip of claim 1 wherein the patterned spacer layer is electrically conductive and configured to function as the integrated resistor of predetermined electrical resistance.

9. The electrochemical-based analytical test strip of claim 9 wherein the patterned spacer layer is formed of an electrically conductive resin.

10. The electrochemical-based analytical test strip of claim 1 wherein the sample-receiving chamber, first electrode and second electrode are configured for the determination of glucose in a whole blood sample.

11. An electrochemical-based analytical test strip for the determination of an analyte in a bodily fluid sample, the electrochemical-based analytical test strip comprising:
an electrically-insulating substrate layer;
a first electrically-conductive layer disposed above the electrically-insulating substrate layer, the first electrically-conductive layer including:
a first electrode portion; and
a first electrical contact pad;
a patterned spacer layer disposed above the first electrically-conductive layer;
a second electrically-conductive layer disposed above the electrically-insulating substrate layer, the second electrically-conductive layer including:
a second electrode portion; and
a second electrical contact pad; and
an integrated resistor configured as an electrically conductive path of predetermined resistance between the first electrically conductive layer and the second electrically conductive layer.

12. A method for determining an analyte in a bodily fluid sample using an electrochemical-based analytical test strip, the method comprising:
measuring an electrical resistance of an integrated resistor of an electrochemical-based analytical test strip using a direct current (DC) bias, wherein the integrated resistor is configured as an electrically conductive path of predetermined resistance between a first electrically conductive layer and a second electrically conductive layer of the electrochemical-based analytical test strip.
detecting an electrochemical response of the electrochemical-based analytical test strip following application of a bodily fluid sample to the electrochemical-based analytical test strip;
compensating the detected electrochemical response for the electrical resistance of the integrated resistor thereby creating a compensated electrochemical response; and
determining an analyte in the bodily fluid sample based on the compensated electrochemical response.

13. The method of claim 12 wherein the measuring includes measuring an electrical resistance in the range of 1.0 k-ohm to 10 M-ohm.

14. The method of claim 13 wherein the measuring includes applying a DC bias, measuring a resulting current and calculating the electrical resistance based on the applied DC bias and the resulting current.

15. The method of claim 12 wherein the detecting includes detecting a transient electrical response.

16. The method of claim 12 wherein a maximum electrical current through the integrated resistor is 1 milli-Amp.

17. The method of claim 12 further including the step of selecting an algorithm for at least one of the detecting, compensating and determining steps based on the measured electrical resistance.

18. The method of claim 12 wherein the compensating step applies compensation based on an applied voltage bias employed during the detecting step.

19. The method of claim 18 wherein detecting step employs a plurality of applied voltage biases and the compensating step applies a plurality of compensations wherein each of the plurality of compensations corresponds to a different applied voltage bias.

20. The method of claim 12 wherein the analyte is glucose and the bodily fluid sample is a whole blood sample.
